(19) United States
(12) Patent Application Publication
(10) Pub. No.: US 2025/0124589 A1
(43) Pub. Date: Apr. 17, 2025

(54) RADIATION IRRADIATION APPARATUS, OPERATION METHOD OF RADIATION IRRADIATION APPARATUS, AND OPERATION PROGRAM

(71) Applicant: FUJIFILM CORPORATION, Tokyo (JP)

(72) Inventors: Tetsuya TSUJI, Tokyo (JP); Ryoji SASADA, Tokyo (JP)

(21) Appl. No.: 18/908,808

(22) Filed: Oct. 8, 2024

(30) Foreign Application Priority Data
Oct. 11, 2023 (JP) 2023-176244

(51) Int. Cl. G06T 7/571; A61B 6/06; A61B 6/40; A61B 6/46

(52) U.S. Cl. CPC G06T 7/571; A61B 6/06; A61B 6/405; A61B 6/463; G06T 2207/30004

(57) ABSTRACT

A radiation irradiation apparatus includes: a radiation source that irradiates an object with radiation; a distance measurement camera that is attached to the radiation source, that includes the object in a field of view, and that generates a distance image in which each pixel value represents a distance; and a processor. The processor transforms the distance image into a three-dimensional point cloud and calculating a distance from a radiation focus to the object based on the transformed three-dimensional point cloud.

### CROSS-REFERENCE TO RELATED APPLICATIONS

The present application claims priority under 35 U.S.C. § 119 to Japanese Patent Application No. 2023-176244, filed on Oct. 11, 2023. The above application is hereby expressly incorporated by reference, in its entirety, into the present application.

### BACKGROUND

### 1. Technical Field

The technology of the present disclosure relates to a radiation irradiation apparatus, an operation method of a radiation irradiation apparatus, and an operation program.

### 2. Description of the Related Art

In a radiation irradiation apparatus that irradiates an object with radiation, it is known that a time-of-flight (ToF) type distance measurement camera is attached to a radiation source, and a distance from the radiation source to the object is measured based on a distance image acquired by the distance measurement camera (see JP2021-191389A).

JP2021-191389A discloses that the distance from the radiation source to the object is measured by disposing the distance measurement camera at a position that can be considered as being approximately the same as a position of a focus of the radiation source (hereinafter, referred to as a radiation focus) in a case in which the radiation source is seen from the object side. In addition, JP2021-191389A discloses that a distance between the radiation focus and the distance measurement camera is measured in advance, and the distance between the radiation focus and the distance measurement camera is added to the distance measured by the distance measurement camera, thereby measuring the distance from the radiation source to the object.

### SUMMARY

In a case in which the radiation focus and the distance measurement camera are misregistrated relative to each other in an optical axis direction, the distance from the radiation source to the object can be measured by measuring a misregistration amount in advance as described above and adding the misregistration amount to the distance measured by the distance measurement camera.

However, since the radiation focus and the distance measurement camera are also misregistrated in a direction orthogonal to the optical axis, it is not possible to accurately measure the distance from the radiation focus to the object by the method disclosed in JP2021-191389A. In order to accurately measure the distance from the radiation focus to the object, it is necessary to measure in advance a three-dimensional misregistration amount between the radiation focus and the distance measurement camera, and to geometrically calculate the distance from the radiation focus to the object in consideration of the three-dimensional misregistration amount. Specifically, it is necessary to geometrically calculate a center position of the radiation on the distance image and to calculate a distance between the center position of the radiation and the radiation focus. Since the center position of the radiation is changed depending on the distance from the radiation focus to the object, it is not easy to calculate the distance from the radiation focus to the object.

An object of the technology of the present disclosure is to provide a radiation irradiation apparatus, an operation method of a radiation irradiation apparatus, and an operation program that can easily and accurately obtain a distance from a radiation focus to an object.

In order to achieve the above-described object, the present disclosure provides a radiation irradiation apparatus comprising: a radiation source that irradiates an object with radiation; a distance measurement camera that is attached to the radiation source, that includes the object in a field of view, and that generates a distance image in which each pixel value represents a distance; and a processor, in which the processor transforms the distance image into a three-dimensional point cloud and calculates a distance from a radiation focus to the object based on the transformed three-dimensional point cloud.

It is preferable that the processor searches for a closest point, which is a point closest to the radiation focus, in the three-dimensional point cloud along an irradiation direction of the radiation from the radiation focus, and calculates a distance from the radiation focus to the closest point detected by the search.

It is preferable that the processor sets a rectangular parallelepiped extending from the radiation focus in an irradiation direction of the radiation, searches for a closest point, which is a point closest to the radiation focus, from a point cloud included in the rectangular parallelepiped in the three-dimensional point cloud, and calculates a distance from the radiation focus to the closest point detected by the search.

It is preferable that the object is a subject, and the processor calculates a first distance, which is a distance from the radiation focus to a surface of the subject.

It is preferable that the object is a surface of a member that is in contact with a subject and that is disposed at a position farther from the radiation source than the subject, and the processor calculates a second distance, which is a distance from the radiation focus to the surface of the member.

It is preferable that the member is a bed, an imaging table, or a radiation image detector.

It is preferable that the processor uses a point cloud included in a region centered on a center position of the radiation in the three-dimensional point cloud, to create a histogram in which the number of points is a first axis and a coordinate of a point in an irradiation direction of the radiation is a second axis, and calculates the second distance by obtaining a value of a coordinate at which the number of points is maximized in the histogram.

It is preferable that the processor changes a size of the region in accordance with a size of the surface of the member.

It is preferable that the object includes a subject and a surface of a member that is in contact with the subject and that is disposed at a position farther from the radiation source than the subject, and the processor performs calculation of a first distance, which is a distance from the radiation focus to a surface of the subject, and calculation of a second distance, which is a distance from the radiation focus to the surface of the member, and calculates a difference between the first distance and the second distance as a body thickness of the subject.

It is preferable that the processor corrects an imaging condition defined by a tube voltage and a tube current-time product based on the body thickness.

It is preferable that the processor gives notification of information on the body thickness by using a notification device, and corrects an imaging condition defined by a tube voltage and a tube current-time product in accordance with an input instruction received by an operation device.

It is preferable that the radiation irradiation apparatus further comprises: an optical camera that is attached to the radiation source and that generates an optical image including the object; and a display device that displays the optical image, in which the radiation source includes a collimator that limits an irradiation field of the radiation, and the processor displays one or both of a center position of the radiation or the irradiation field in a superimposed manner on the optical image.

It is preferable that, in a case in which aperture information of the collimator is included, the processor determines a size of the irradiation field on the optical image based on the aperture information.

It is preferable that, in a case in which aperture information of the collimator is not included, the processor determines a size of the irradiation field on the optical image based on aperture information of the collimator, which is associated with an imaging menu.

The present disclosure provides an operation method of a radiation irradiation apparatus including a radiation source that irradiates an object with radiation, and a distance measurement camera that is attached to the radiation source, that includes the object in a field of view, and that generates a distance image in which each pixel value represents a distance, the operation method comprising: transforming, via a processor, the distance image into a three-dimensional point cloud and calculating a distance from a radiation focus to the object based on the transformed three-dimensional point cloud.

The present disclosure provides an operation program for operating a radiation irradiation apparatus including a radiation source that irradiates an object with radiation, and a distance measurement camera that is attached to the radiation source, that includes the object in a field of view, and that generates a distance image in which each pixel value represents a distance, the operation program causing a processor to execute a process comprising: transforming the distance image into a three-dimensional point cloud and calculating a distance from a radiation focus to the object based on the transformed three-dimensional point cloud.

According to the technology of the present disclosure, it is possible to provide the radiation irradiation apparatus, the operation method of the radiation irradiation apparatus, and the operation program that can easily and accurately obtain the distance from the radiation focus to the object.

### BRIEF DESCRIPTION OF THE DRAWINGS

Exemplary embodiments according to the technique of the present disclosure will be described in detail based on the following figures, wherein:

FIG. 1 is a view showing an example of a radiography system,

FIG. 2 is a view of a radiation source seen in a direction orthogonal to a radiation irradiation direction,

FIG. 3 is a view of the radiation source seen in a direction parallel to the radiation irradiation direction,

FIG. 4 is a view showing a configuration example of a console,

FIG. 5 is a view showing an example of a function related to determination of an imaging condition formed in a CPU,

FIG. 6 is a view schematically showing an example of a distance image,

FIG. 7 is a view schematically showing an example of a three-dimensional point cloud,

FIG. 8 is a view showing a configuration example of a body thickness calculation unit,

FIG. 9 is a view showing an example of calculation processing of an SSD and an SID,

FIG. 10 is a view showing an example of a histogram created in a case in which the SID is calculated,

FIG. 11 is a view showing an example of a center position of radiation on the distance image,

FIG. 12 is a view showing an example of icons representing a plurality of physical builds,

FIG. 13 is a view showing another example of the function related to the determination of the imaging condition formed in the CPU,

FIG. 14 is a view showing another example of the calculation processing of the SSD,

FIG. 15 is a view showing an example of displaying the center position and an irradiation field of the radiation in a superimposed manner on an optical image,

FIG. 16 is a view showing examples of an imaging menu and aperture information, and

FIG. 17 is a view showing an example of displaying a frame representing an outer shape of an electronic cassette in a superimposed manner on the optical image.

### DETAILED DESCRIPTION

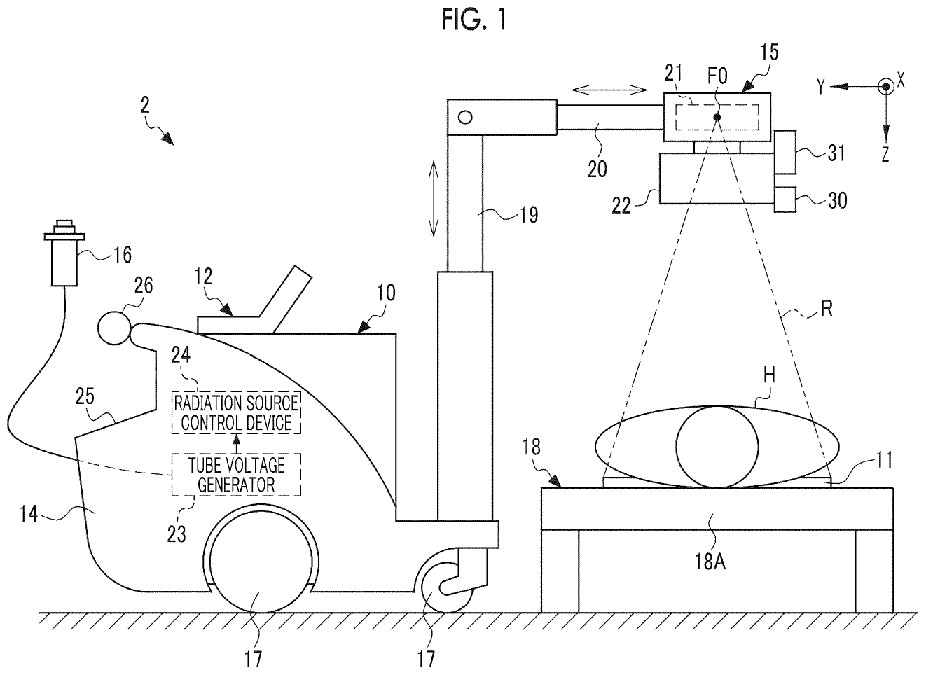

FIG. 1 shows an example of a radiography system. As shown in FIG. 1, a radiography system 2 is a system that performs radiography of a subject H using radiation (for example, X-rays) R, and includes a mobile radiation irradiation apparatus 10 and an electronic cassette 11. In the present embodiment, the subject His a subject under examination, such as a patient. The radiation irradiation apparatus 10 includes a console 12, a carriage unit 14, a radiation source 15, and an irradiation switch 16. Four wheels 17 are attached to a lower part of the carriage unit 14. The wheels 17 allow the radiation irradiation apparatus 10 to be used for radiography during medical rounds, in which the subject His imaged with the radiation while the radiation irradiation apparatus 10 circulates through a sickroom in a medical facility. Therefore, the radiation irradiation apparatus 10 is also referred to as a mobile X-ray unit.

FIG. 1 shows a state in which chest front surface imaging of the subject H lying in a supine posture on a bed 18 in the sickroom is performed. In this case, the electronic cassette 11 is inserted between the subject H and a top plate 18A of the bed 18 by an operator such as a radiologist. That is, in the present example, the electronic cassette 11 is not used in a state of being stored in a holder of an imaging table installed in a radiography room, and is removed from the holder and used.

The radiation source 15 is attached to the carriage unit 14 via a first arm 19 and a second arm 20. The first arm 19 extends in a vertical direction from a front part of the carriage unit 14 and can be expanded and contracted in the vertical direction. A height position of the radiation source 15 is changed by the extension and contraction of the first arm 19. The first arm 19 is rotatable about a vertical axis as a rotation axis.

The second arm 20 extends in a horizontal direction from a distal end of the first arm 19. The second arm 20 can be expanded and contracted in the horizontal direction. A horizontal position of the radiation source 15 is changed by the extension and contraction of the second arm 20.

The radiation source 15 includes a radiation tube 21 and a collimator 22, and irradiates an object with radiation. In the present embodiment, the object is surfaces of the subject H and the bed 18. It should be noted that the bed 18 is an example of a “member that is in contact with the subject and that is disposed at a position farther from the radiation source than the subject” according to the technology of the present disclosure.

The radiation tube 21 is provided with a filament, a target, a grid electrode, and the like (none of which is shown). A tube voltage is applied between the filament as a cathode and the target as an anode. The filament releases thermoelectrons toward a radiation focus F0 on the target in accordance with the applied tube voltage. The radiation focus F0 on the target emits the radiation R due to the collision of the thermoelectrons from the filament. The grid electrode is disposed between the filament and the target. The grid electrode changes a flow rate of the thermoelectrons from the filament toward the target in accordance with the applied voltage. The flow rate of the thermoelectrons from the filament toward the target is referred to as a tube current.

The collimator 22 limits an irradiation field of the radiation R emitted from the radiation focus F0 of the radiation tube 21. The collimator 22 is configured to partially shield the radiation R with, for example, a square stop 22A (see FIG. 3) formed of four shielding plates such as lead. The collimator 22 changes the irradiation field of the radiation R by changing a position of each of the shielding plates to change an aperture of the stop 22A.

A radiation source control device 23 and a tube voltage generator 24 are built in the carriage unit 14. The radiation source control device 23 is connected to the console 12 in a wirelessly communicable manner. In addition, the radiation source control device 23 controls an operation of the tube voltage generator 24. Further, the irradiation switch 16 is connected to the radiation source control device 23. The radiation source control device 23 controls an operation of the radiation source 15 in accordance with various instruction signals from the irradiation switch 16. The irradiation switch 16 is operated in a case in which the operator instructs the radiation source 15 to start the irradiation with the radiation R. The irradiation switch 16 is attachably and detachably attached to the carriage unit 14.

An imaging condition is set in the radiation source control device 23. For example, the imaging condition is a condition defined by the tube voltage applied to the radiation tube 21 and a tube current-time product (product of the tube current and an irradiation time). For example, the tube voltage is a value with “kV” as a unit, and the tube current-time product is a value with “mAs” as a unit. In a case in which the instruction to start the irradiation with the radiation R is given by the operation of the irradiation switch 16, the radiation source control device 23 operates the tube voltage generator 24 based on the set imaging condition to perform the irradiation with the radiation R via the radiation tube 21. The tube voltage generator 24 generates the tube voltage by boosting the input voltage with a transformer. The tube voltage generated by the tube voltage generator 24 is supplied to the radiation tube 21 via a voltage cable (not shown).

A cassette storage portion 25 and a handle 26 are provided at a rear part of the carriage unit 14. The cassette storage portion 25 stores the electronic cassette 11. There are a plurality of types of the electronic cassettes 11 having vertical and horizontal sizes, such as 14×14 inches, 14×17 inches, and 17×17 inches. The cassette storage portion 25 can store a plurality of electronic cassettes 11 regardless of the type. Further, the cassette storage portion 25 has a function of charging a battery of the stored electronic cassette 11.

The electronic cassette 11 is a portable radiation image detector that detects the radiation R transmitted through the subject H, to generate a radiation image. The electronic cassette 11 includes a detection panel in which a plurality of pixels accumulating charges corresponding to the radiation R are arranged in a two-dimensional matrix. The detection panel is also referred to as a flat panel detector (FPD).

The handle 26 is gripped by the operator in a case in which moving the carriage unit 14. The console 12 is a computer including a processor and a memory, and may be incorporated into the carriage unit 14, or may be a laptop personal computer or the like.

A camera unit 30 and a monitor 31 are attached to the radiation source 15. For example, the camera unit 30 and the monitor 31 are attached to an outside of a housing of the collimator 22. A distance measurement camera 32 and an optical camera 33 (see FIG. 3) are built in the camera unit 30. The operations of the distance measurement camera 32, the optical camera 33, and the monitor 31 are controlled by the console 12.

The distance measurement camera 32 includes the object in a field of view FOV, and generates a distance image in which each pixel represents a distance. The field of view FOV is a region that includes the irradiation field in which the irradiation with the radiation R is performed by the radiation source 15. The distance image is a two-dimensional image including a plurality of pixel values, and each pixel value thereof represents the distance. The distance image is also referred to as a depth image.

The distance measurement camera 32 is, for example, a ToF camera. In this case, the distance measurement camera 32 includes a light transmission unit and a light reception unit (not shown), and is configured to acquire distance information to the object by measuring a time from the emission of the measurement light via the light transmission unit toward the object to the reception of the measurement light, which is reflected by the object and returned, via the light reception unit. The measurement light is, for example, infrared laser light. Further, the measurement light may be pulse light, or may be intensity-modulated continuous light. In a case in which the continuous light is used as the measurement light, the above-described time can be measured by obtaining a phase difference between the measurement light emitted from the light transmission unit and the measurement light received by the light reception unit.

It should be noted that the distance measurement camera 32 is not limited to the ToF camera, and may also be a stereo camera or the like that measures a distance using parallax information. The distance measurement camera 32 need only be a camera that can measure the distance information to generate the distance image.

The optical camera 33 generates an optical image by optically imaging the object included in the field of view FOV. For example, the optical camera 33 is a digital camera including an imaging element, such as a complementary metal-oxide-semiconductor (CMOS) sensor, in which photodiodes are arranged in a two-dimensional array. For example, the field of view FOV of the optical camera 33 is a region including the irradiation field in which the irradiation with the radiation R is performed by the radiation source 15, similarly to the field of view FOV of the distance measurement camera 32. For example, the optical image is a color image including red (R), green (G), and blue (B) pixels. The optical image may be a moving image.

The monitor 31 displays the imaging condition, the optical image generated by the optical camera 33, and the like. The monitor 31 is, for example, a display such as a liquid crystal display. The monitor 31 may be provided with an operation device such as a touch panel. The optical image is used for positioning the subject H with respect to the irradiation field of the radiation R. In addition, the imaging condition is determined in detail by, for example, correcting an imaging condition, which is provisionally determined based on a selected imaging menu, based on a body thickness of the subject H calculated based on the distance image.

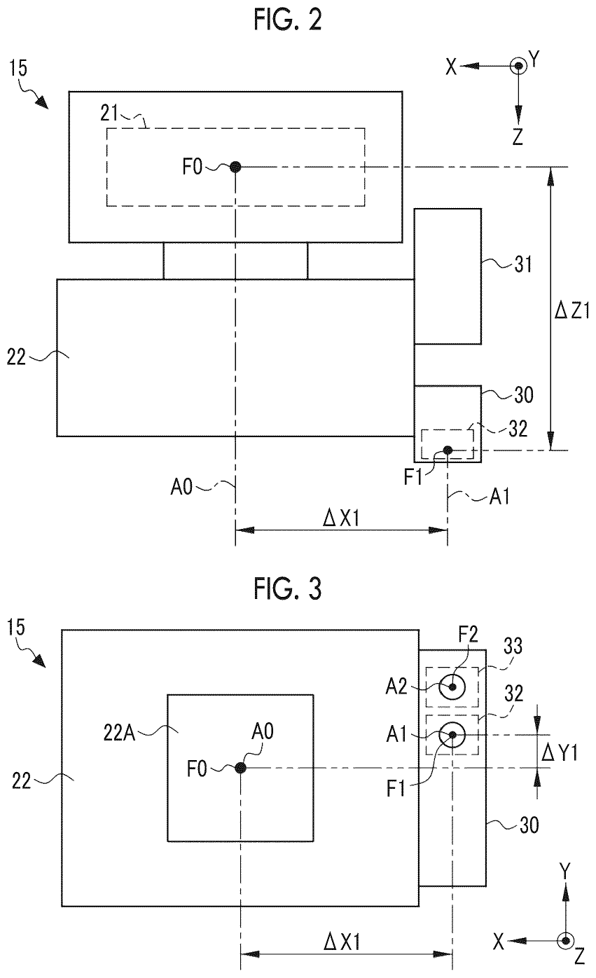

FIGS. 2 and 3 show a configuration of the radiation source 15. FIG. 2 is a view of the radiation source 15 seen in a direction orthogonal to an irradiation direction of the radiation R. FIG. 3 is a view of the radiation source 15 seen in a direction parallel to the irradiation direction of the radiation R. In the present disclosure, the irradiation direction of the radiation R is defined as a Z direction, one direction orthogonal to the Z direction is defined as an X direction, and a direction orthogonal to both the Z direction and the X direction is defined as a Y direction.

An optical axis A0 of the radiation R is a straight line passing through a center position of the irradiation field of the radiation R (hereinafter, also referred to as the center position of the radiation R) from the radiation focus F0, and is parallel to the irradiation direction (Z direction) of the radiation R. The distance measurement camera 32 is disposed such that an optical axis A1 thereof is parallel to the optical axis A0 of the radiation R. The optical axis A1 of the distance measurement camera 32 is a straight line passing through the center of the field of view FOV from a camera focus F1. The camera focus F1 is a rear-side focus located on a focal plane of the distance measurement camera 32.

In the same manner, the optical camera 33 is disposed such that an optical axis A2 thereof is parallel to the optical axis A0 of the radiation R. The optical axis A2 of the optical camera 33 is a straight line passing through the center of the field of view FOV from a camera focus F2. The camera focus F2 is a rear-side focus located on a focal plane of the optical camera 33.

The radiation focus F0 and the camera focus F1 are misregistrated in three-dimensional directions, that is, the X direction, the Y direction, and the Z direction. Hereinafter, the misregistration amounts of the camera focus F1 in the X direction, the Y direction, and the Z direction with respect to the radiation focus F0 are denoted by ΔX1, ΔY1, and ΔZ1, respectively.

In the same manner, the radiation focus F0 and the camera focus F2 are misregistrated in three-dimensional directions, that is, the X direction, the Y direction, and the Z direction. Although not shown in FIGS. 2 and 3, the misregistration amounts of the camera focus F2 in the X direction, the Y direction, and the Z direction with respect to the radiation focus F0 are denoted by ΔX2, ΔY2, and ΔZ2, respectively. For example, the optical camera 33 is disposed to satisfy a relationship of ΔX1=ΔX2, ΔY1+ΔY2, and ΔZ1=ΔZ2.

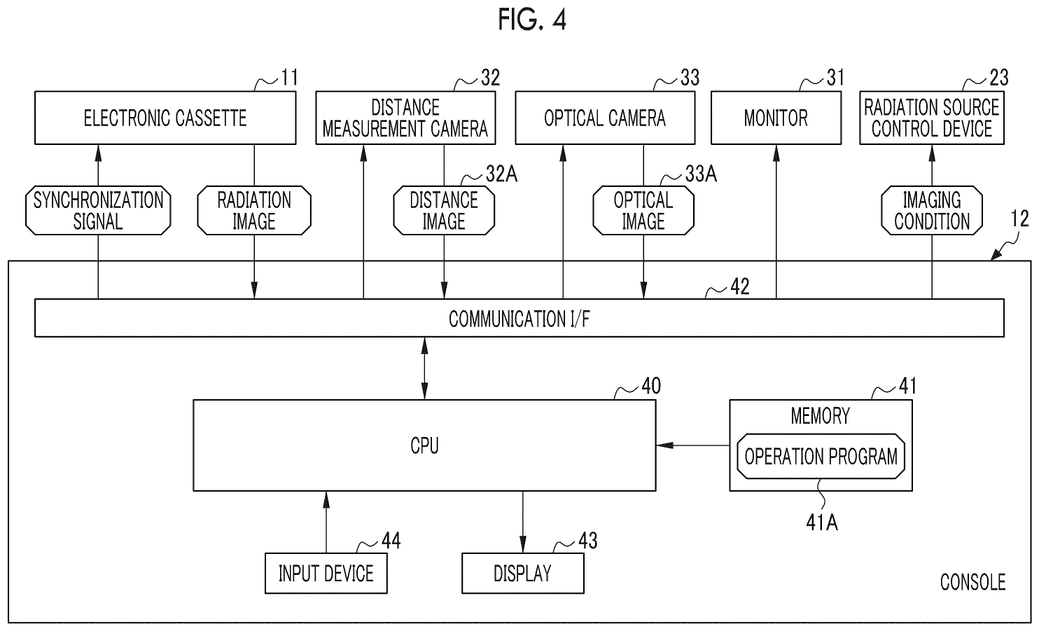

FIG. 4 shows a configuration example of the console 12. The console 12 comprises a central processing unit (CPU) 40, a memory 41, a communication interface (I/F) 42, a display 43, and an input device 44. The display 43 is a liquid crystal display and the like, and displays various screens. The input device 44 includes a keyboard, a mouse, and the like, and receives operation instructions from the operator.

The memory 41 is a storage device such as a flash memory built in or connected to the CPU 40. The memory 41 stores an operation program 41A, various data, and the like. The CPU 40 executes the processing based on the operation program 41A stored in the memory 41. As a result, the CPU 40 integrally controls the respective units of the computer. The CPU 40 is an example of a “processor” according to the technology of the present disclosure. The communication I/F 42 performs transmission control of various information with external devices such as the electronic cassette 11.

The CPU 40 displays a plurality of types of imaging menus in a selectable manner on the display 43. The imaging menu defines an imaging technique including an imaging part of the subject H, an imaging posture of the subject H, and an imaging direction of the subject H as one set, such as “chest, upright posture, front surface” and “abdomen, upright posture, front surface”. Examples of the imaging part include a head, a neck, an abdomen, a waist, a shoulder, an elbow, a hand, a knee, and an ankle, in addition to the chest. Examples of the imaging posture include a decubitus posture and a sitting posture, in addition to the upright posture. Examples of the imaging direction include a back surface and a side surface, in addition to the front surface. The operator operates the input device 44 to select one imaging menu from among the plurality of types of imaging menus. As a result, the CPU 40 receives the selected imaging menu.

The CPU 40 acquires a distance image 32A output from the distance measurement camera 32 and an optical image 33A output from the optical camera 33 via the communication I/F 42 before starting the radiography. The CPU 40 calculates a body thickness BT of the subject H based on the distance image 32A in addition to the selected imaging menu, and determines the imaging condition based on the calculated body thickness BT. The CPU 40 transmits the determined imaging condition to the radiation source control device 23 via the communication I/F 42.

In addition, the CPU 40 outputs the optical image 33A to the monitor 31 via the communication I/F 42, thereby displaying the optical image 33A on the monitor 31.

In addition, in a case in which the instruction to start the irradiation with the radiation R is given to the radiation source control device 23 via the irradiation switch 16, the CPU 40 receives an irradiation start signal from the radiation source control device 23 indicating that the irradiation with the radiation R is started. In a case in which the irradiation start signal is received, the CPU 40 transmits a synchronization signal indicating that the irradiation with the radiation R is started, to the electronic cassette 11. Further, the CPU 40 receives an irradiation end signal indicating that the irradiation with the radiation R ends from the radiation source control device 23. In a case in which the irradiation end signal is received, the CPU 40 transmits a synchronization signal indicating that the irradiation with the radiation R ends, to the electronic cassette 11.

In a case in which the synchronization signal indicating that the irradiation with the radiation R is started is received from the console 12, the electronic cassette 11 causes the detection panel to start an accumulation operation. In addition, in a case in which the synchronization signal indicating that the irradiation with the radiation R ends is received from the console 12, the electronic cassette 11 causes the detection panel to start a readout operation.

The CPU 40 receives the radiation image from the electronic cassette 11 via the communication I/F 42. The CPU 40 performs various types of image processing on the radiation image, and then displays the radiation image on the display 43 to provide the radiation image for viewing by the operator.

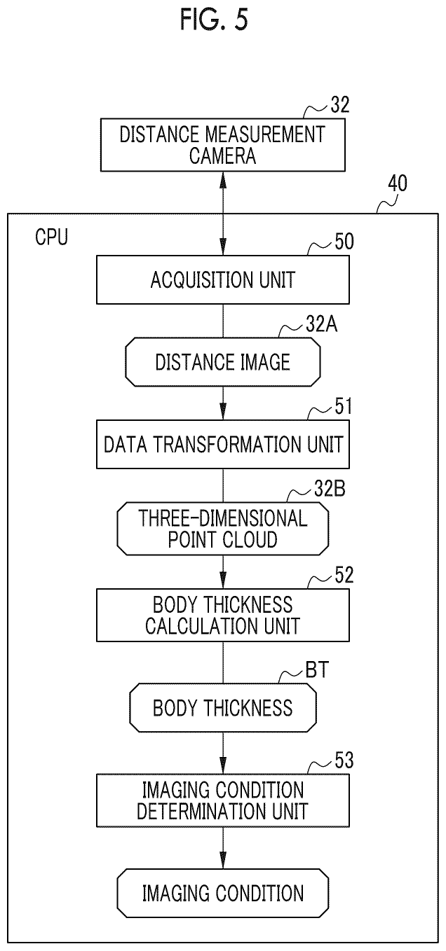

FIG. 5 shows an example of a function related to the determination of the imaging condition formed in the CPU 40. The CPU 40 executes the processing based on the operation program 41A, to function as an acquisition unit 50, a data transformation unit 51, a body thickness calculation unit 52, and an imaging condition determination unit 53.

The acquisition unit 50 controls the distance measurement camera 32 to acquire the distance image 32A output from the distance measurement camera 32. For example, as shown in FIG. 1, the acquisition unit 50 acquires the distance image 32A in accordance with the operator's operation on the operation device such as the input device 44 in a state in which the subject H is lying in a supine posture on the bed 18. The field of view FOV of the distance measurement camera 32 includes at least the subject H and a part of the bed 18.

The data transformation unit 51 transforms the distance image 32A acquired by the acquisition unit 50 into a three-dimensional point cloud 32B using a known method. The three-dimensional point cloud 32B is data obtained by transforming each pixel value included in the distance image 32A into a point in a three-dimensional space, and is a set of the points in three-dimensional space.

The body thickness calculation unit 52 calculates the body thickness BT of the subject H based on the three-dimensional point cloud 32B in the three-dimensional space.

The imaging condition determination unit 53 determines the imaging condition by correcting the imaging condition, which is provisionally determined based on the selected imaging menu, based on the body thickness BT calculated by the body thickness calculation unit 52. Specifically, the imaging condition determination unit 53 estimates a physical build of the subject H from among a plurality of physical builds based on the body thickness BT, and corrects the imaging condition based on the estimated physical build. For example, the imaging condition determination unit 53 increases the tube current and/or the tube current-time product as the physical build is increased. For example, the plurality of physical builds are three physical builds, “large”, “medium”, and “small”. It should be noted that the imaging condition determination unit 53 may correct the imaging condition based on an SID calculated by an SID calculation unit 52B described below in addition to the body thickness BT. For example, the tube current and/or the tube current-time product may be increased as the SID is increased.

The imaging condition determined by the imaging condition determination unit 53 is transmitted to the radiation source control device 23. The radiation source control device 23 operates the tube voltage generator 24 based on the received imaging condition.

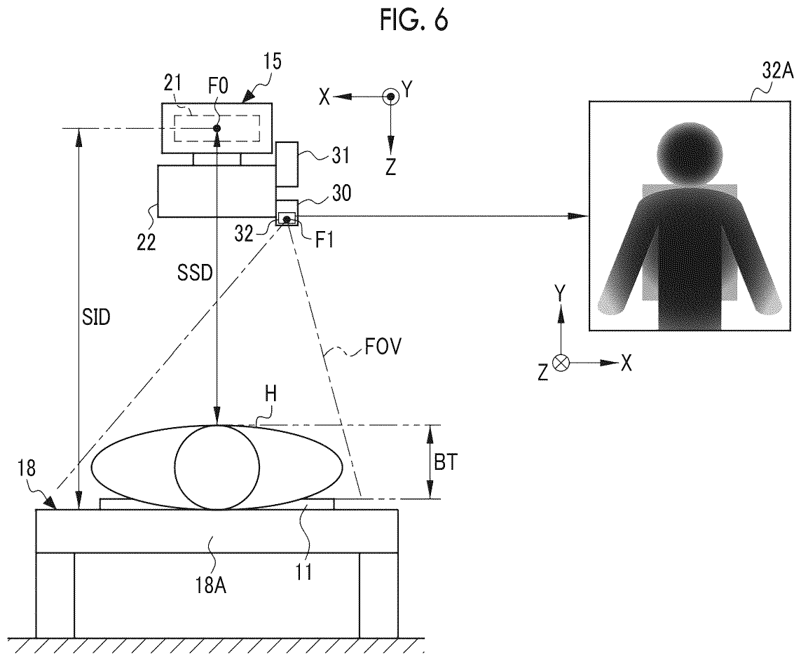

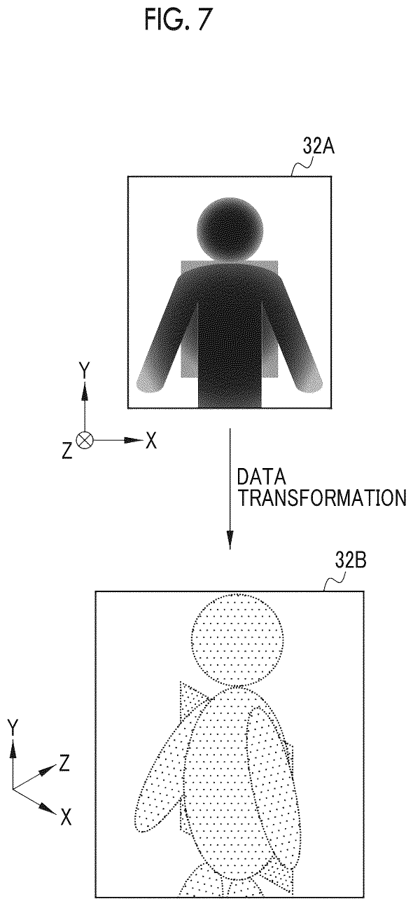

FIG. 6 schematically shows an example of the distance image 32A output from the distance measurement camera 32. FIG. 7 schematically shows an example of the three-dimensional point cloud 32B transformed by the data transformation unit 51. For example, the three-dimensional point cloud 32B is represented by coordinates in which an X axis parallel to the X direction, a Y axis parallel to the Y direction, and a Z axis parallel to the Z direction are used as coordinate axes. It should be noted that, as shown in FIGS. 2 and 3, since the positional relationship between the radiation focus F0 and the camera focus F1 is known, the coordinates of the radiation focus F0 in the three-dimensional point cloud 32B are specified based on the camera focus F1. For example, in a case in which the coordinates of the radiation focus F0 are (X_{F0}, Y_{F0}, Z_{F0}) and the coordinates of the camera focus F1 are (X_{F1}, Y_{F1}, Z_{F1}), a relationship of (X_{F0}, Y_{F0}, Z_{F0})=(X_{F1}+ΔX1, Y_{F1}−ΔY1, Z_{F1}−ΔZ1) holds true. It should be noted that ΔX1>0, ΔY1>0, and ΔZ1>0.

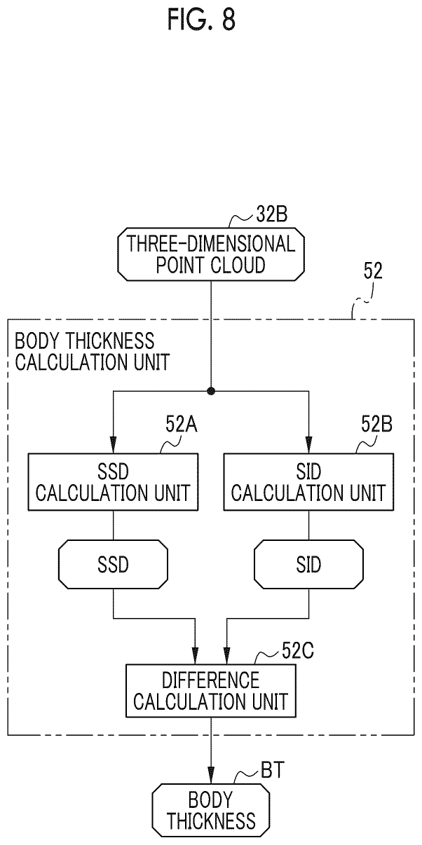

FIG. 8 shows a configuration example of the body thickness calculation unit 52. The body thickness calculation unit 52 includes a source-to-surface distance (SSD) calculation unit 52A that calculates an SSD, a source-to-image distance (SID) calculation unit 52B that calculates the SID, and a difference calculation unit 52C. As shown in FIG. 6, the SSD is a distance from the radiation focus F0 to the surface of the subject H, and corresponds to a “first distance” according to the technology of the present disclosure. In the present embodiment, the SID is a distance from the radiation focus F0 to the surface of the top plate 18A of the bed 18, and corresponds to a “second distance” according to the technology of the present disclosure. It should be noted that the SSD and the SID are lengths along the Z direction. Further, the SSD is a length in the Z direction with the radiation focus F0 as a starting point.

The SSD calculation unit 52A calculates the SSD based on the three-dimensional point cloud 32B in the three-dimensional space. The SID calculation unit 52B calculates the SID based on the three-dimensional point cloud 32B in the three-dimensional space. The difference calculation unit 52C calculates a difference between the SID calculated by the SID calculation unit 52B and the SSD calculated by the SSD calculation unit 52A, as the body thickness BT. Specifically, the difference calculation unit 52C calculates the body thickness BT by using a relational expression BT=SID−SSD.

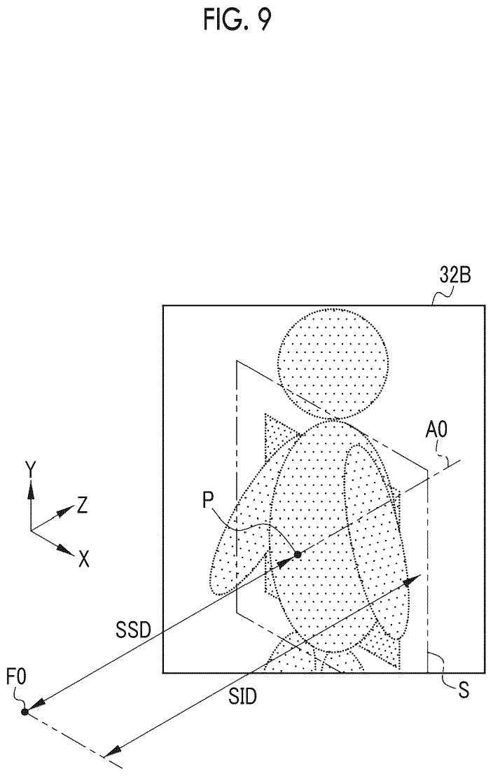

FIG. 9 shows an example of calculation processing of the SSD and the SID. As shown in FIG. 9, the SSD calculation unit 52A searches for a point P (hereinafter referred to as a closest point P) closest to the radiation focus F0 in the three-dimensional point cloud 32B from the radiation focus F0 along the Z direction (that is, the irradiation direction of the radiation R). Then, the SSD calculation unit 52A calculates a distance from the radiation focus F0 to the closest point P detected by the search, as the SSD. The closest point P is a point on the surface of the subject H at the center position of the radiation R.

The SID calculation unit 52B sets a predetermined region S centered on the center position of the radiation R (that is, the center position of the irradiation field). In addition, the SID calculation unit 52B detects a plane (that is, an XY plane) perpendicular to the Z direction from the point cloud included in the region S in the three-dimensional point cloud 32B, which is on the farther side from the radiation focus F0 with the closest point P as a starting point and which is detected by the SSD calculation unit 52A in a case in which the SSD is calculated, and calculates the difference between the Z coordinate of the detected plane and the radiation focus F0, as the SID. For example, the region S is a rectangular region that is larger than a planar shape of the electronic cassette 11 and is smaller than the surface of the top plate 18A.

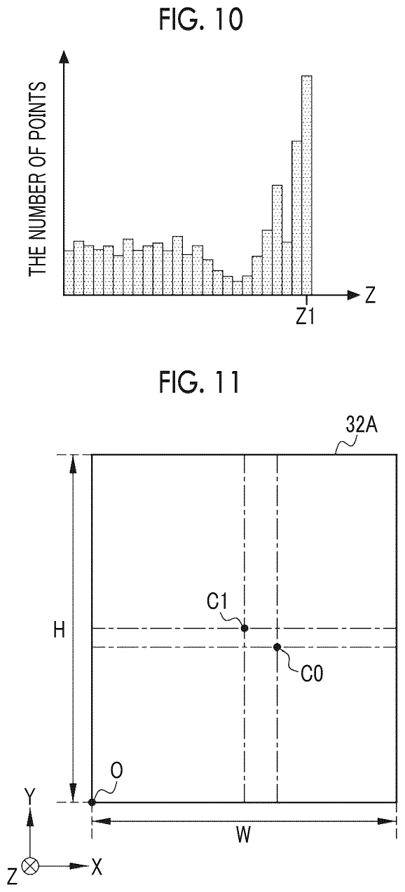

Specifically, the SID calculation unit 52B uses the three-dimensional point cloud 32B included in the region S to create a histogram, as shown in FIG. 10, in which the number of points is a vertical axis and the Z coordinate (that is, the coordinate of the point in the irradiation direction of the radiation R) is a horizontal axis, and obtains a value Zp of the Z coordinate at which the number of points is maximized. Since the value Zp is estimated as the Z coordinate of the surface of the top plate 18A, the SID calculation unit 52B calculates the SID by using a relational expression SID=Zp−Z_{F0}. It should be noted that the vertical axis is an example of a “first axis” according to the technology of the present disclosure. The horizontal axis is an example of a “second axis” according to the technology of the present disclosure.

It should be noted that a size of the region S may be changed in accordance with a surface size of the top plate 18A. It is preferable that the size of the region S is set such that the region S does not include a region outside the top plate 18A. In addition, the size of the region S may be appropriately changed in accordance with a size of the electronic cassette 11 or the like. The size of the region S may be, for example, equal to or smaller than the size of the electronic cassette 11. In this case, a surface of the electronic cassette 11 is detected as the plane perpendicular to the Z direction. That is, the SID may be a distance in the Z direction from the radiation focus F0 to the surface of the electronic cassette 11. In this case, the electronic cassette 11 is an example of a “member that is in contact with the subject and that is disposed at a position farther from the radiation source than the subject” according to the technology of the present disclosure.

As described above, according to the technology of the present disclosure, the distance image 32A acquired by the distance measurement camera 32 attached to the radiation source 15 is transformed into the three-dimensional point cloud 32B, and the distance from the radiation focus F0 to the object is calculated based on the transformed three-dimensional point cloud 32B, so that the distance from the radiation focus F0 to the object can be easily and accurately obtained.

FIG. 11 shows an example of the center position C0 of the radiation R on the distance image 32A. In FIG. 11, C1 indicates a center position of the distance image 32A, that is, a position through which the optical axis A1 passes. W indicates a length of the distance image 32A in the X direction. H indicates a length of the distance image 32A in the Y direction. The units of the length W and the length H are the number of pixels. O is an origin.

In a case in which a coordinate of the center position C0 of the radiation R with respect to the origin O in the X direction is denoted by Xc, and a coordinate in the Y direction is denoted by Yc, the coordinates Xc and Yc are represented by Expressions (1A) and (1B), respectively.

$$Xc = ( W / 2 ) \times ( 1 + {α}_{X} / d ) \qquad ( 1 A )$$

$$Yc = ( H / 2 ) \times ( 1 - {α}_{Y} / d ) \qquad ( 1 B )$$

Here, d is a distance from the camera focus F1 to an imaging surface, and is represented by, for example, Expression (2).

$$d = S I D - Δ Z 1 \qquad ( 2 )$$

α_{X} and α_{Y} are represented by Expressions (3A) and (3B).

$${α}_{X} = Δ X 1 / tan ( {ω}_{X} / 2 ) \qquad ( 3 A )$$

$${α}_{Y} = Δ Y 1 / tan ( {ω}_{Y} / 2 ) \qquad ( 3 B )$$

Here, ωX is an angle of view of the distance measurement camera 32 in the X direction. ωY is an angle of view of the distance measurement camera 32 in the Y direction. ΔX1, ΔY1, and ΔZ1 are misregistration amounts between the radiation focus F0 and the camera focus F1.

In a case in which the center position C0 of the radiation R on the distance image 32A can be specified, the distance from the radiation focus F0 to the object can be obtained based on the distance image 32A. However, as is clear from Expressions (1A) and (1B), the center position C0 of the radiation R on the distance image 32A is changed following a hyperbolic function in accordance with the distance d, and thus it is not easy to specify the center position C0. In particular, in the radiography system 2 in which the mobile radiation irradiation apparatus 10 is used as in the above-described embodiment, the change is made each time the radiation source 15 and the subject H are positioned by the operator, the distance d varies, and thus the center position C0 of the radiation R varies. As described above, in a case in which the center position C0 of the radiation R varies, it is difficult to accurately specify the center position C0 of the radiation R on the distance image 32A, and thus it is not possible to accurately obtain the distance from the radiation focus F0 to the object. On the other hand, in the three-dimensional point cloud 32B obtained by transforming the distance image 32A, the coordinates of the radiation focus F0 can be easily specified based on the misregistration amounts ΔX1, ΔY1, and ΔZ1, so that the distance from the radiation focus F0 to the object can be easily and accurately obtained.

Hereinafter, various modification examples of the above-described embodiment will be described.

In the above-described embodiment, the CPU 40 corrects the imaging condition based on the body thickness BT calculated by the body thickness calculation unit 52. However, the imaging condition may also be corrected in accordance with the input instruction received by the operation device, by giving notification of the information related to the calculated body thickness BT via a notification device. The notification device is, for example, a display device such as the display 43 or the monitor 31. The operation device is the input device 44 or the like.

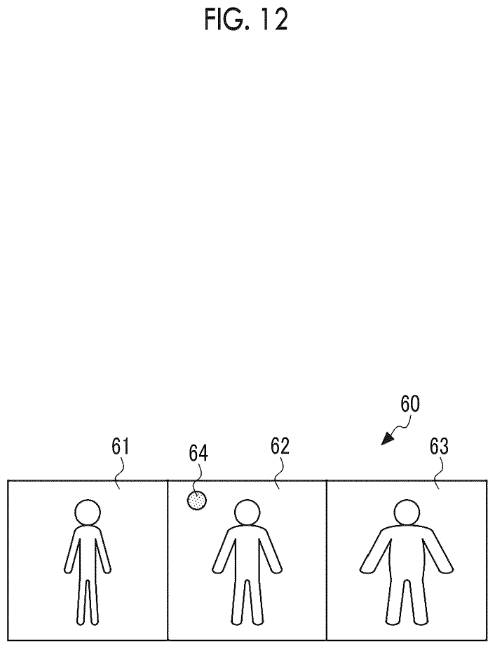

For example, as shown in FIG. 12, the CPU 40 displays icons 60 representing a plurality of physical builds 61 to 63 on the display device, such as the display 43 and the monitor 31, and allows the operator to select any one of the plurality of physical builds 61 to 63 using the operation device, such as the input device 44. One of the plurality of physical builds 61 to 63 is a physical build estimated based on the body thickness BT calculated by the body thickness calculation unit 52. The physical build estimated based on the body thickness BT is an example of “information on the body thickness” according to the technology of the present disclosure.

In the example shown in FIG. 12, the CPU 40 displays a mark 64 for identifying the physical build 62 estimated based on the body thickness BT calculated by the body thickness calculation unit 52, to propose the selection of the physical build 62 to the operator. The CPU 40 corrects the imaging condition in accordance with the physical build selected by the operator, determines the corrected imaging condition, and transmits the determined imaging condition to the radiation source control device 23. It should be noted that the operator can also select a physical build other than the proposed physical build.

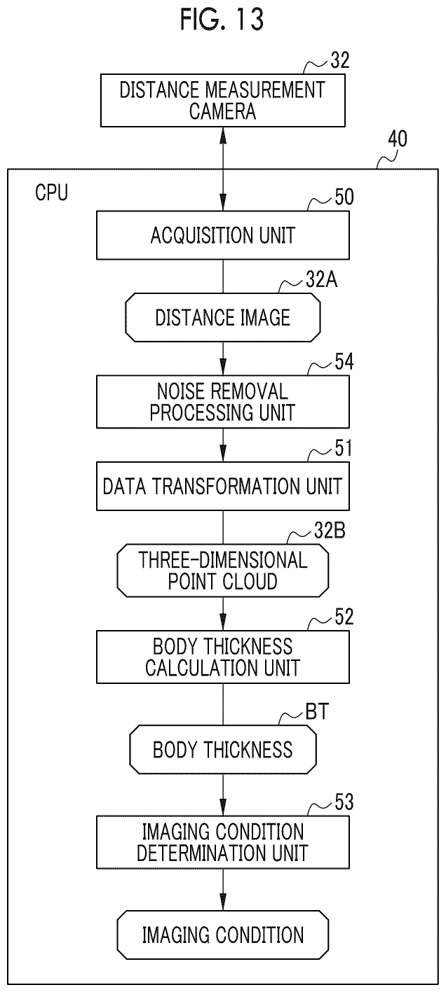

In the above-described embodiment, the data transformation unit 51 of the CPU 40 transforms the distance image 32A acquired from the distance measurement camera 32 into the three-dimensional point cloud 32B. However, an image obtained by performing noise removal processing on the distance image 32A may also be transformed into the three-dimensional point cloud 32B. Specifically, as shown in FIG. 13, a noise removal processing unit 54 is added between the acquisition unit 50 and the data transformation unit 51. The noise removal processing unit 54 removes noise from the distance image 32A by performing filtering processing, such as median filtering, on the distance image 32A, and inputs the distance image 32A on which the noise removal processing has be performed to the data transformation unit 51. As described above, by performing noise removal processing on the distance image 32A, the noise in the three-dimensional point cloud 32B is removed, and the accuracy of calculating the SSD and the SID is improved. As a result, the accuracy of calculating the body thickness BT is improved.

In the above-described embodiment, since the radiation irradiation direction and the optical axis A1 of the distance measurement camera 32 are parallel to each other, the SSD calculation unit 52A detects the closest point P by performing the search in the three-dimensional point cloud 32B along the radiation irradiation direction (that is, the Z direction) from the radiation focus F0. However, in reality, the radiation irradiation direction and the optical axis A1 may not be parallel to each other, and it is conceivable that the accuracy of detecting the closest point P is reduced in a case in which the search is performed from the radiation focus F0 in the radiation irradiation direction in a case in which the radiation irradiation direction and the optical axis A1 are not parallel to each other.

Therefore, a misregistration angle between the radiation irradiation direction and the optical axis A1 may be obtained in advance by calibration, the coordinates of the three-dimensional point cloud 32B may be transformed by a rotation operation corresponding to the obtained misregistration angle, and the closest point P may be detected to calculate the SSD by using the three-dimensional point cloud 32B after the coordinate transformation to perform the search along the Z direction after the transformation. Specifically, the coordinate transformation is performed by the rotation operation on the three-dimensional point cloud 32B from a three-dimensional coordinate system in which the direction of the optical axis A1 is the Z axis to a three-dimensional coordinate system in which the radiation irradiation direction is the Z axis, based on the misregistration angle obtained by the calibration.

It should be noted that, for example, the calibration is performed by acquiring the distance image 32A in a state in which the center position C0 of the radiation R is matched with a predetermined position on the surface of the top plate 18A of the bed 18 without disposing the subject H and by setting the SID to a predetermined value. The misregistration angle between the radiation irradiation direction and the optical axis A1 can be obtained by measuring the misregistration amount of the center position C0 specified in the acquired distance image 32A from a geometrically determined position in the X direction and the Y direction.

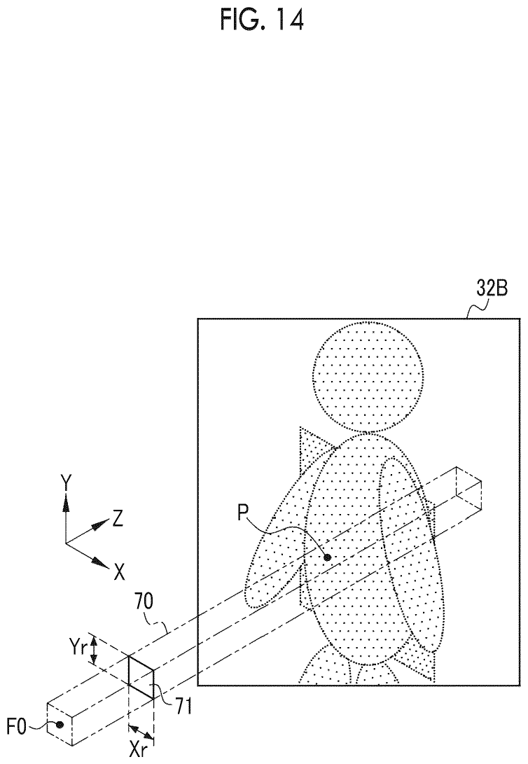

In addition, the closest point P may be detected by the following method. As shown in FIG. 14, the SSD calculation unit 52A sets a rectangular parallelepiped 70 extending from the radiation focus F0 in the radiation irradiation direction, and searches for the closest point P from the point cloud included in the rectangular parallelepiped 70 in the three-dimensional point cloud 32B. In the point cloud included in the rectangular parallelepiped 70, the point closest to the radiation focus F0 may be set as the closest point P. In addition, an average value of the coordinates of the point cloud included in the rectangular parallelepiped 70 may be obtained, and a point in which the average value is the coordinate may be used as the closest point P. Specifically, an average value of the X coordinates, an average value of the Y coordinates, and an average value of the Z coordinates of the point cloud included in the rectangular parallelepiped 70 are calculated, and the point in which the calculated average value of the X coordinates, the calculated average value of the Y coordinates, and the calculated average value of the Z coordinates are the X coordinate, the Y coordinate, and the Z coordinate, respectively, is set as the closest point P.

A cross section 71 of the rectangular parallelepiped 70 is defined based on an upper limit value of an angle between the radiation irradiation direction and the optical axis A1, and an upper limit value of the SID. The cross section 71 is a rectangular surface having a side parallel to the X direction and a side parallel to the Y direction. A length Xr of the side parallel to the X direction and a length Yr of the side parallel to the Y direction are represented by Expressions (4A) and (4B), respectively.

$$Xr = {SID}_{max} \times sin ( {θ}_{X max} ) \qquad ( 4 A )$$

$$Yr = {SID}_{max} \times sin ( {θ}_{Y max} ) \qquad ( 4 B )$$

Here, SIDₘₐₓ is an upper limit value of the assumed SID. θ_{Xmax} is a X-direction component of an upper limit of the angle formed between the radiation irradiation direction and the optical axis A1. θ_{Ymax} is a Y-direction component of an upper limit of the angle formed between the radiation irradiation direction and the optical axis A1.

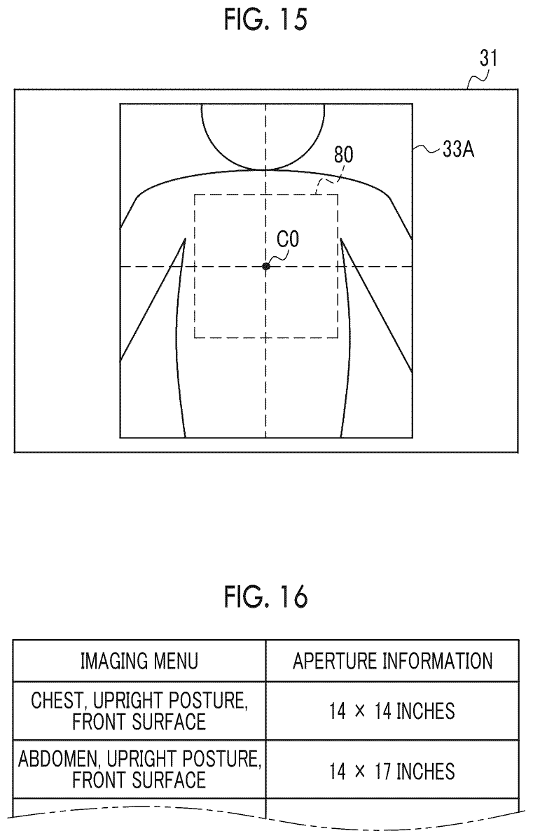

In addition, in the above-described embodiment, the CPU 40 displays the optical image 33A output from the optical camera 33 on the monitor 31. As shown in FIG. 15, the CPU 40 may display the center position C0 of the radiation R in a superimposed manner on the optical image 33A displayed on the monitor 31. The center position C0 of the radiation R corresponds to the closest point P. The CPU 40 uses the misregistration amounts ΔX2, ΔY2, and ΔZ2 between the radiation focus F0 and the camera focus F2 and the SSD calculated by the SSD calculation unit 52A, to calculate the coordinates of the center position C0 of the radiation R on the optical image 33A, and displays a mark indicating the center position C0 of the radiation R at the calculated coordinates.

Specifically, the X coordinate Xc′ and the Y coordinate Yc′ of the center position C0 of the radiation R on the optical image 33A are represented by Expressions (5A) and (5B), respectively.

$$Xc ’ = ( W ’ / 2 ) \times ( 1 + {β}_{X} / d ’ ) \qquad ( 5 A )$$

$$Yc ’ = ( H ’ / 2 ) \times ( 1 - {β}_{Y} / d ’ ) \qquad ( 5 B )$$

Here, d′ is a distance from the camera focus F2 to the imaging surface, and is represented by, for example, Expression (6). W′ indicates a length of the optical image 33A in the X direction. H′ indicates a length of the optical image 33A in the Y direction.

$$d ’ = S I D - Δ Z 2 \qquad ( 6 )$$

β_{X} and β_{Y} are represented by Expressions (7A) and (7B).

$${β}_{X} = Δ X 2 / tan ( {Ω}_{X} / 2 ) \qquad ( 7 A )$$

$${β}_{Y} = Δ Y 2 / tan ( {Ω}_{Y} / 2 ) \qquad ( 7 B )$$

Here, Ω_{X} is an angle of view of the optical camera 33 in the X direction. Ω_{Y} is an angle of view of the optical camera 33 in the Y direction.

In addition, the CPU 40 may display the irradiation field 80 of the radiation R in a superimposed manner on the optical image 33A displayed on the monitor 31, with the center position C0 of the radiation R as the center. In a case in which the radiation irradiation apparatus 10 has aperture information of the stop 22A of the collimator 22, the CPU 40 determines the size of the irradiation field 80 to be displayed in a superimposed manner on the optical image 33A based on the aperture information. For example, the aperture information of the stop 22A is stored in the memory 41, and the CPU 40 determines the size of the irradiation field 80 on the optical image 33A based on the aperture information read out from the memory 41. In addition, in a case in which the radiation irradiation apparatus 10 does not have the aperture information, the CPU 40 determines the size of the irradiation field 80 on the optical image 33A based on the aperture information associated with the imaging menu selected by the operator. For example, as shown in FIG. 16, the imaging menu and the aperture information are associated with each other.

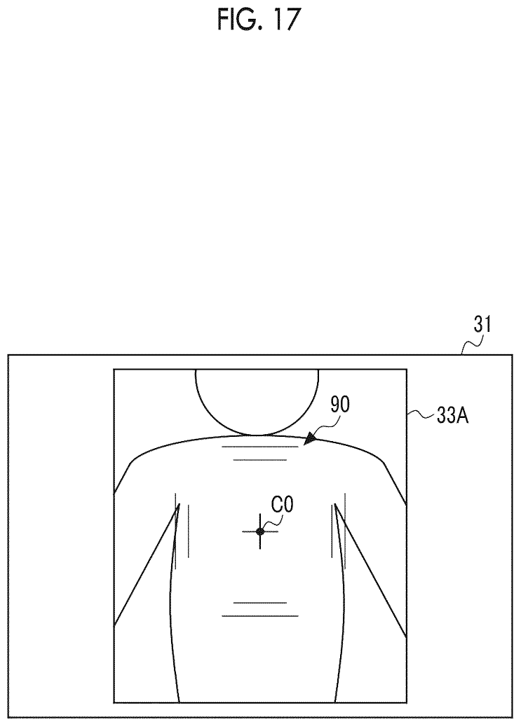

In addition, the CPU 40 may display a frame representing an outer shape of the electronic cassette 11 in a superimposed manner on the optical image 33A displayed on the monitor 31, with the center position C0 of the radiation R as the center. For example, as shown in FIG. 17, the CPU 40 displays a frame 90 representing the outer shape of the plurality of types of electronic cassettes 11 that can be used in the radiography system 2, with the center position C0 of the radiation R as the center. For example, the frame 90 represents an outer edge portion of the electronic cassette 11 having the size of 14×14 inches and an outer edge portion of the electronic cassette 11 having size of 17×17 inches. Since the electronic cassette 11 may be disposed on a back surface of the subject H and may not appear in the optical image 33A, the operator can recognize the outer shape of the electronic cassette 11 during the positioning of the subject H by displaying the frame representing the outer shape of the electronic cassette 11 in a superimposed manner on the optical image 33A.

It is preferable that the size of the frame 90 is represented as the size on the surface of the subject H on the optical image 33A. For example, in a case in which a length of the frame 90 in the X direction is denoted by D′, an actual length of the electronic cassette 11 in the X direction is denoted by D, and a distance from the camera focus F2 to the surface of the subject His denoted by k (=SSD−ΔZ2), the length D′ of the frame 90 in the X direction is represented by Expression (8).

$$D ’ = D \times \{ W ’ / ( 2 \times k \times tan ( {Ω}_{X} / 2 ) ) \} \qquad ( 8 )$$

Here, W′ is a length of the optical image 33A in the X direction. Ω_{X} is an angle of view of the optical camera 33 in the X direction. A length of the frame 90 in the Y direction can also be obtained by the same expression.

In addition, it is preferable that the irradiation field 80 shown in FIG. 15 is also represented as the size on the surface of the subject H on the optical image 33A. In a case in which a solid angle of the radiation R in the X direction of the irradiation field, which is determined by the aperture of the stop 22A of the collimator 22, is denoted by Φ_{X}, a length L of the irradiation field of the radiation R on the surface of the subject H in the X direction is represented by Expression (9).

$$L = 2 \times S S D \times tan ( {Φ}_{X} / 2 ) \qquad ( 9 )$$

A length L′ of the irradiation field 80 displayed on the optical image 33A in the X direction is represented by Expression (10) in the same manner as in Expression (8) by using the length L calculated by Expression (9).

$$L ’ = L \times \{ W ’ / ( 2 \times k \times tan ( {Ω}_{X} / 2 ) ) \} \qquad ( 10 )$$

A length of the irradiation field 80 displayed on the optical image 33A in the Y direction can also be obtained by the same expression.

In addition, in the above-described embodiment, the technology of the present disclosure is described by using, as an example, the radiography system 2 including the mobile radiation irradiation apparatus 10. The technology of the present disclosure is not limited to the mobile radiography system, and can also be applied to a radiography system in which the radiation irradiation apparatus is installed in the radiography room or the like. For example, the radiation irradiation apparatus according to the present disclosure may be movably supported by a support portion via a ceiling traveling apparatus or a floor traveling apparatus. In this case, the subject is disposed with respect to the imaging table such as an upright imaging table or a decubitus imaging table. In addition, the electronic cassette is stored in a holder provided on the imaging table. In this case, the imaging table is an example of a “member that is in contact with the subject and that is disposed at a position farther from the radiation source than the subject” according to the technology of the present disclosure.

A hardware configuration of the processor in the above-described embodiment can be variously modified as follows. The processor includes a CPU, a programmable logic device (PLD), a dedicated electric circuit, or a combination of the CPU, the PLD, and the dedicated electric circuit. As is well known, the CPU is a general-purpose processor that functions as various processing units by executing software (that is, a program). The PLD is a processor of which a circuit configuration is changeable after manufacturing, and is, for example, a field programmable gate array (FPGA). The dedicated electric circuit is a processor of which a circuit configuration is specially designed for executing specific processing, such as an application specific integrated circuit (ASIC). Further, the hardware structure of these various processors is, more specifically, an electric circuit (circuitry) in which circuit elements such as semiconductor elements are combined.

The above-described embodiment and the respective modification examples can be combined in two or more combinations as long as there is no contradiction.

The technology of the present disclosure is not limited to the above-described embodiment and the respective modification examples, and it goes without saying that various configurations can be employed without departing from the gist of the present disclosure. Further, the technology of the present disclosure extends to a computer-readable storage medium non-transitorily storing the program, in addition to the program.

The following technology can be understood by the above description.

### Supplementary Note 1

A radiation irradiation apparatus comprising: a radiation source that irradiates an object with radiation; a distance measurement camera that is attached to the radiation source, that includes the object in a field of view, and that generates a distance image in which each pixel value represents a distance; and a processor, in which the processor transforms the distance image into a three-dimensional point cloud and calculates a distance from a radiation focus to the object based on the transformed three-dimensional point cloud.

### Supplementary Note 2

The radiation irradiation apparatus according to supplementary note 1, in which the processor searches for a closest point, which is a point closest to the radiation focus, in the three-dimensional point cloud along an irradiation direction of the radiation from the radiation focus, and calculates a distance from the radiation focus to the closest point detected by the search.

### Supplementary Note 3

The radiation irradiation apparatus according to supplementary note 1, in which the processor sets a rectangular parallelepiped extending from the radiation focus in an irradiation direction of the radiation, searches for a closest point, which is a point closest to the radiation focus, from a point cloud included in the rectangular parallelepiped in the three-dimensional point cloud, and calculates a distance from the radiation focus to the closest point detected by the search.

### Supplementary Note 4

The radiation irradiation apparatus according to any one of supplementary notes 1 to 3, in which the object is a subject, and the processor calculates a first distance, which is a distance from the radiation focus to a surface of the subject.

### Supplementary Note 5

The radiation irradiation apparatus according to any one of supplementary notes 1 to 3, in which the object is a surface of a member that is in contact with a subject and that is disposed at a position farther from the radiation source than the subject, and the processor calculates a second distance, which is a distance from the radiation focus to the surface of the member.

### Supplementary Note 6

The radiation irradiation apparatus according to supplementary note 5, in which the member is a bed, an imaging table, or a radiation image detector.

### Supplementary Note 7

The radiation irradiation apparatus according to supplementary note 5 or 6, in which the processor uses a point cloud included in a region centered on a center position of the radiation in the three-dimensional point cloud, to create a histogram in which the number of points is a first axis and a coordinate of a point in an irradiation direction of the radiation is a second axis, and calculates the second distance by obtaining a value of a coordinate at which the number of points is maximized in the histogram.

### Supplementary Note 8

The radiation irradiation apparatus according to supplementary note 7, in which the processor changes a size of the region in accordance with a size of the surface of the member.

### Supplementary Note 9

The radiation irradiation apparatus according to any one of supplementary notes 1 to 3, in which the object includes a subject and a surface of a member that is in contact with the subject and that is disposed at a position farther from the radiation source than the subject, and the processor performs calculation of a first distance, which is a distance from the radiation focus to a surface of the subject, and calculation of a second distance, which is a distance from the radiation focus to the surface of the member, and calculates a difference between the first distance and the second distance as a body thickness of the subject.

### Supplementary Note 10

The radiation irradiation apparatus according to supplementary note 9, in which the processor corrects an imaging condition defined by a tube voltage and a tube current-time product based on the body thickness.

### Supplementary Note 11

The radiation irradiation apparatus according to supplementary note 9, in which the processor gives notification of information on the body thickness by using a notification device, and corrects an imaging condition defined by a tube voltage and a tube current-time product in accordance with an input instruction received by an operation device.

### Supplementary Note 12

The radiation irradiation apparatus according to any one of supplementary notes 1 to 11, further comprising: an optical camera that is attached to the radiation source and that generates an optical image including the object; and a display device that displays the optical image, in which the radiation source includes a collimator that limits an irradiation field of the radiation, and the processor displays one or both of a center position of the radiation or the irradiation field in a superimposed manner on the optical image.

### Supplementary Note 13

The radiation irradiation apparatus according to supplementary note 12, in which, in a case in which aperture information of the collimator is included, the processor determines a size of the irradiation field on the optical image based on the aperture information.

### Supplementary Note 14

The radiation irradiation apparatus according to supplementary note 12, in which, in a case in which aperture information of the collimator is not included, the processor determines a size of the irradiation field on the optical image based on aperture information of the collimator, which is associated with an imaging menu.

What is claimed is:

1. A radiation irradiation apparatus comprising:
a radiation source that irradiates an object with radiation;
a distance measurement camera that is attached to the radiation source, that includes the object in a field of view, and that generates a distance image in which each pixel value represents a distance; and
a processor,
wherein the processor transforms the distance image into a three-dimensional point cloud and calculates a distance from a radiation focus to the object based on the transformed three-dimensional point cloud.

2. The radiation irradiation apparatus according to claim 1,
wherein the processor
searches for a closest point, which is a point closest to the radiation focus, in the three-dimensional point cloud along an irradiation direction of the radiation from the radiation focus, and
calculates a distance from the radiation focus to the closest point detected by the search.

3. The radiation irradiation apparatus according to claim 1,
wherein the processor
sets a rectangular parallelepiped extending from the radiation focus in an irradiation direction of the radiation,
searches for a closest point, which is a point closest to the radiation focus, from a point cloud included in the rectangular parallelepiped in the three-dimensional point cloud, and
calculates a distance from the radiation focus to the closest point detected by the search.

4. The radiation irradiation apparatus according to claim 1,
wherein the object is a subject, and
the processor calculates a first distance, which is a distance from the radiation focus to a surface of the subject.

5. The radiation irradiation apparatus according to claim 1,
wherein the object is a surface of a member that is in contact with a subject and that is disposed at a position farther from the radiation source than the subject, and
the processor calculates a second distance, which is a distance from the radiation focus to the surface of the member.

6. The radiation irradiation apparatus according to claim 5,
wherein the member is a bed, an imaging table, or a radiation image detector.

7. The radiation irradiation apparatus according to claim 5,
wherein the processor
uses a point cloud included in a region centered on a center position of the radiation in the three-dimensional point cloud, to create a histogram in which the number of points is a first axis and a coordinate of a point in an irradiation direction of the radiation is a second axis, and
calculates the second distance by obtaining a value of a coordinate at which the number of points is maximized in the histogram.

8. The radiation irradiation apparatus according to claim 7,
wherein the processor changes a size of the region in accordance with a size of the surface of the member.

9. The radiation irradiation apparatus according to claim 1,
wherein the object includes a subject and a surface of a member that is in contact with the subject and that is disposed at a position farther from the radiation source than the subject, and
the processor
performs calculation of a first distance, which is a distance from the radiation focus to a surface of the subject, and calculation of a second distance, which is a distance from the radiation focus to the surface of the member, and
calculates a difference between the first distance and the second distance as a body thickness of the subject.

10. The radiation irradiation apparatus according to claim 9,
wherein the processor corrects an imaging condition defined by a tube voltage and a tube current-time product based on the body thickness.

11. The radiation irradiation apparatus according to claim 9,
wherein the processor
gives notification of information on the body thickness by using a notification device, and
corrects an imaging condition defined by a tube voltage and a tube current-time product in accordance with an input instruction received by an operation device.

12. The radiation irradiation apparatus according to claim 1, further comprising:
an optical camera that is attached to the radiation source and that generates an optical image including the object; and
a display device that displays the optical image,
wherein the radiation source includes a collimator that limits an irradiation field of the radiation, and
the processor displays one or both of a center position of the radiation or the irradiation field in a superimposed manner on the optical image.

13. The radiation irradiation apparatus according to claim 12,
wherein, in a case in which aperture information of the collimator is included, the processor determines a size of the irradiation field on the optical image based on the aperture information.

14. The radiation irradiation apparatus according to claim 12,
wherein, in a case in which aperture information of the collimator is not included, the processor determines a size of the irradiation field on the optical image based on aperture information of the collimator, which is associated with an imaging menu.

15. An operation method of a radiation irradiation apparatus including a radiation source that irradiates an object with radiation, and a distance measurement camera that is attached to the radiation source, that includes the object in a field of view, and that generates a distance image in which each pixel value represents a distance, the operation method comprising:
transforming, via a processor, the distance image into a three-dimensional point cloud and calculates a distance from a radiation focus to the object based on the transformed three-dimensional point cloud.

16. A non-transitory computer-readable storage medium storing an operation program for operating a radiation irradiation apparatus including a radiation source that irradiates an object with radiation, and a distance measurement camera that is attached to the radiation source, that includes the object in a field of view, and that generates a distance image in which each pixel value represents a distance, the operation program causing a processor to execute a process comprising:
transforming the distance image into a three-dimensional point cloud and calculating a distance from a radiation focus to the object based on the transformed three-dimensional point cloud.
